(19) United States
(12) Patent Application Publication
(10) Pub. No.: US 2017/0255002 A1
(43) Pub. Date: Sep. 7, 2017

(54) IMAGE PICKUP OPTICAL SYSTEM AND IMAGE PICKUP UNIT OF ENDOSCOPE, AND ENDOSCOPE

(71) Applicant: FUJIFILM Corporation, Tokyo (JP)

(72) Inventor: Takashi YASHIRO, Ashigarakami-gun (JP)

(73) Assignee: FUJIFILM Corporation, Tokyo (JP)

(21) Appl. No.: 15/604,667

(22) Filed: May 25, 2017

Related U.S. Application Data
(63) Continuation of application No. PCT/JP2015/081641, filed on Nov. 10, 2015.

(30) Foreign Application Priority Data
Dec. 4, 2014 (JP) 2014-245927

(51) Int. Cl. G02B 23/24; G03B 17/08; G03B 17/12; A61B 1/04; H04N 5/225

(52) U.S. Cl. CPC G02B 23/2484; A61B 1/04; H04N 5/2256; G02B 23/243; G03B 17/12; G03B 17/08; H04N 2005/2255

(57) ABSTRACT

An image pickup optical system of an endoscope includes: a first optical member group that includes an objective optical member disposed closest to a subject side and an image-side optical member that is disposed adjacent to an image side of the objective optical member; a first lens barrel that holds the first optical member group; a second optical member group that is disposed on an image side of the first optical member group; and a second lens barrel that holds the second optical member group, as defined herein.

### CROSS REFERENCE TO RELATED APPLICATION

This is a continuation of International Application No. PCT/JP2015/081641 filed on Nov. 10, 2015, and claims priority from Japanese Patent Application No. 2014-245927 filed on Dec. 4, 2014, the entire disclosures of which are incorporated herein by reference.

### BACKGROUND OF THE INVENTION

1. Field of the Invention

The present invention relates to an image pickup optical system and an image pickup unit of an endoscope, and an endoscope.

2. Description of the Related Art

An image pickup unit of an endoscope includes an image pickup optical system that includes a plurality of combinations of optical members, such as lenses, and an image pickup element. In order to ensure image pickup performance, the image pickup optical system and the image pickup element are typically integrated so as not to be separable from each other.

An optical member, which is disposed closest to a subject side and is exposed to the outside, among the plurality of optical members of the image pickup optical system is likely to be stained or damaged with use. In a case in which a stain or a damage occurs on an optical member exposed to the outside, the entire image pickup unit including an image pickup element should be replaced in the case of an image pickup unit of which an image pickup optical system and an image pickup element are integrated so as not to be separable from each other. For this reason, repair cost is increased.

In contrast, an image pickup unit of an endoscope of which only some optical members provided on the subject side are adapted to be replaceable is also known.

An image pickup unit of an endoscope disclosed in JP1998-99260A (JP-H10-99260A) includes: a first optical frame member that holds a subject-side first optical member group including an observation window exposed to the outside and an optical member disposed adjacent to the observation window among a plurality of optical members of an image pickup optical system; and a second optical frame member that holds an image pickup element and a second optical member group excluding the first optical member group among the plurality of optical members of the image pickup optical system. The first optical frame member is fitted to the second optical frame member, and is detachably mounted on the second optical frame member. In a case in which a stain or a damage occurs on the observation window, the first optical frame member is separated from the second optical frame member and the first optical frame member and the first optical member group are replaced all together.

An image pickup unit of an endoscope disclosed in JP1999-352414A (JP-H11-352414A) includes: a lens frame that holds a subject-side first optical member group including a first lens exposed to the outside and a second lens disposed adjacent to the first lens among a plurality of optical members of an image pickup optical system; and an image pickup element frame that holds an image pickup element and a second optical member group excluding the first optical member group among the plurality of optical members of the image pickup optical system. However, the lens frame is bonded and fixed to the image pickup element frame. In a case in which a stain or a damage occurs on the first lens exposed to the outside in the image pickup unit disclosed in JP1999-352414A (JP-H11-352414A), the first lens, which is stained or damaged, is taken off to be removed from the lens frame and is replaced with a new first lens. The second lens is also adapted to be removable from the lens frame, and is replaced in conjunction with the first lens.

### SUMMARY OF THE INVENTION

In the image pickup unit of an endoscope disclosed in JP1998-99260A (JP-H10-99260A), the first optical member group includes the observation window and the optical member disposed adjacent to the observation window, and the observation window and the optical member are assembled to the first optical frame member so as to be spaced apart from each other in the direction of the optical axis. For this reason, since the tolerance of the first optical frame member affects the positional relationship of the observation window, the optical member, and the second optical member group, the adjustment of a focus of the image pickup optical system is necessary when the first optical frame member and the first optical member group are replaced.

Since the first and second lenses are individually assembled to the lens frame in the image pickup unit of an endoscope disclosed in JP1999-352414A (JP-H11-352414A), the adjustment of a focus of the image pickup optical system is also necessary when the first and second lenses are replaced.

The invention has been made in consideration of the above-mentioned circumstances, and an object of the invention is to provide an image pickup optical system and an image pickup unit of an endoscope, and an endoscope of which some optical member groups disposed on the subject side can be replaced and which can reduce time and effort for the adjustment of a focus accompanying the replacement of the optical member groups.

An image pickup optical system of an endoscope according to an aspect of the invention comprises a first optical member group that includes a plurality of optical members including an objective optical member disposed closest to a subject side, a first lens barrel that holds the first optical member group, a second optical member group that is disposed on an image side of the first optical member group, and a second lens barrel that holds the second optical member group. The plurality of optical members of the first optical member group are in contact with each other in a direction of an optical axis; the first and second lens barrels are fitted to each other in the direction of the optical axis so that the optical member, which is disposed closest to an image side, of the first optical member group is in contact with the second lens barrel; and a space is provided on an image side of an image-side end of the first lens barrel and on a subject side of a subject-side end of the second lens barrel in the direction of the optical axis in a state in which the optical member, which is disposed closest to the image side, of the first optical member group is in contact with the second lens barrel, and the first and second lens barrels are bonded to each other so as to be separable from each other.

Further, an image pickup unit of an endoscope according to another aspect of the invention comprises the image pickup optical system, and an image pickup element that is provided at an image-side end portion of the second lens barrel of the image pickup optical system.

Furthermore, an endoscope according to a still another aspect of the invention comprises the image pickup unit.

According to the invention, it is possible to provide an image pickup optical system and an image pickup unit of an endoscope, and an endoscope of which some optical member groups disposed on the subject side can be replaced and which can reduce time and effort for the adjustment of a focus accompanying the replacement of the optical member groups.

### BRIEF DESCRIPTION OF THE DRAWINGS

FIG. 1 is a view showing the structure of an example of an endoscope that is used to illustrate an embodiment of the invention.

FIG. 2 is a view showing the structure of an image pickup unit of the endoscope of FIG. 1.

FIG. 3 is a view showing the structure of another example of the image pickup unit of the endoscope that is used to illustrate the embodiment of the invention.

FIG. 4 is a view showing the structure of still another example of the image pickup unit of the endoscope that is used to illustrate the embodiment of the invention.

### EXPLANATION OF REFERENCES

1: endoscope

5: image pickup unit

6: image pickup optical system

7: image pickup element

10: first optical member group

11: first lens barrel

12: second optical member group

13: second lens barrel

20: objective optical member

21: optical member

23: optical member

30: fitting portion

31: fitting portion

32: contact portion

33: flange portion

### DESCRIPTION OF THE PREFERRED EMBODIMENTS

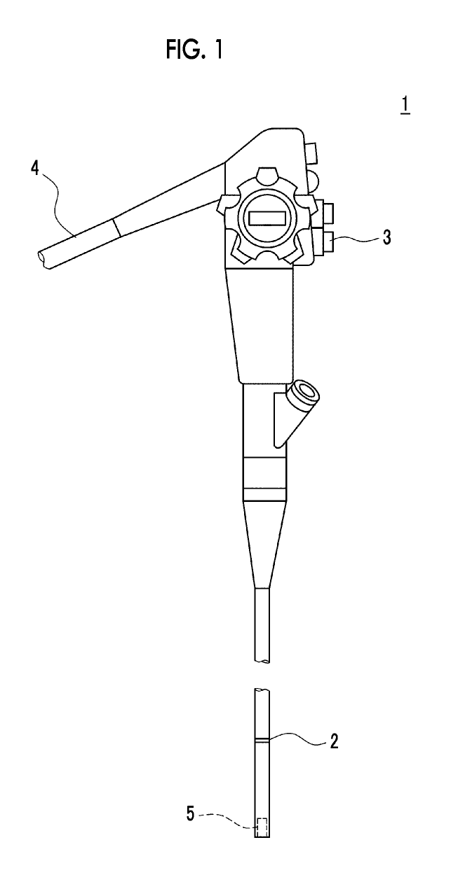

FIG. 1 shows the structure of an example of an endoscope that is used to illustrate an embodiment of the invention.

The endoscope 1 includes an insertion part 2, an operation part 3 that is connected to the insertion part 2, and a universal cord 4 that extends from the operation part 3.

An illumination optical system that emits illumination light for illuminating a subject and an image pickup unit 5 that picks up the image of the subject are provided at the tip portion of the insertion part 2.

Since a connector is provided at the end of the universal cord 4, the endoscope 1 is connected to a light source device, which generates illumination light, and a processor device, which processes image signals acquired by the image pickup unit 5, through the connector provided at the end of the universal cord 4.

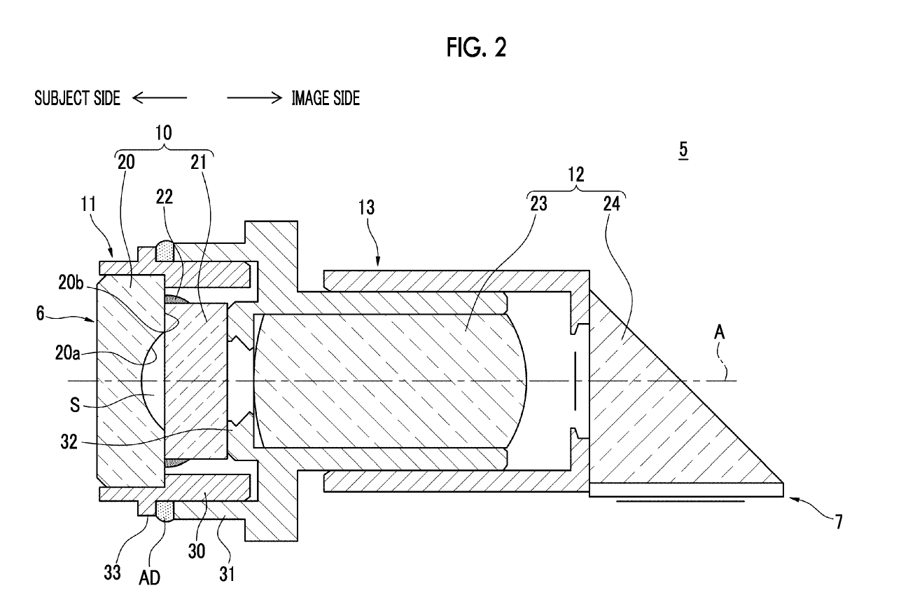

FIG. 2 shows the structure of the image pickup unit 5.

The image pickup unit 5 includes: an image pickup optical system 6 that is formed of a combination of a plurality of optical members, such as lenses; and an image pickup element 7, such as a charge coupled device (CCD) image sensor or a complementary metal oxide semiconductor (CMOS) image sensor.

The image pickup optical system 6 includes a first optical member group 10 that includes some optical members disposed on a subject side, a first lens barrel 11 that holds the first optical member group 10, a second optical member group 12 that is disposed on the image side of the first optical member group 10, and a second lens barrel 13 that holds the second optical member group 12.

The first optical member group 10 includes a plurality of optical members including an objective optical member 20 that is disposed closest to the subject side among the plurality of optical members of the image pickup optical system 6 and is exposed to the outside and an optical member 21 that is disposed adjacent to the image side of the objective optical member 20. The first optical member group 10 may include other optical members that are disposed on the image side of the optical member 21 in addition to the objective optical member 20 and the optical member 21.

The objective optical member 20 and the optical member 21 are fixed to the first lens barrel 11 in a state in which the objective optical member 20 and the optical member 21 are in contact with each other in the direction of an optical axis of the image pickup optical system 6.

Typically, as in the example shown in FIG. 2, an optical surface 20a that is formed in the shape of a concave curved surface and a flat surface 20b, which surrounds the optical surface 20a and is substantially perpendicular to the optical axis A of the image pickup optical system 6, are provided on the image-side surface of the objective optical member 20, that is, on the surface that faces the optical member 21. The optical member 21 is disposed so that the subject-side surface of the optical member 21 is in contact with the flat surface 20b of the objective optical member 20. The subject-side surface of the optical member 21 has been a flat surface substantially perpendicular to the optical axis A in the example shown in FIG. 2, but may be, for example, a convex curved surface or the like.

Further, the optical member 21 is bonded to the flat surface 20b of the objective optical member 20 by using an appropriate bonding material 22, such as an adhesive, and the objective optical member 20 is fitted to and held by the first lens barrel 11. Accordingly, the objective optical member 20 and the optical member 21 are fixed to the first lens barrel 11 in a state in which the objective optical member 20 and the optical member 21 are in contact with each other.

A space S is formed between the objective optical member 20 and the optical member 21 by the optical surface 20a that is formed in the shape of a concave curved surface. Preferably, the objective optical member 20 and the optical member 21 are bonded to each other so that the space S is made airtight. The objective optical member 20 is cooled by, for example, water, which is sent to remove dirt adhering to the subject-side surface exposed to the outside, or the like. However, since the space S is made airtight, the infiltration of moisture into the space S is suppressed. As a result, the generation of condensation can be suppressed.

In the shown example in which the optical member 21 is bonded to the objective optical member 20 by using the bonding material 22, the bonding material 22 may be provided over the entire circumference of the optical member 21 to bond the objective optical member 20 to the optical member 21 so that the space S is made airtight.

The second optical member group 12 includes one or more optical members, and is appropriately adapted according to the design of the image pickup optical system 6. In the shown example, the second optical member group 12 includes an optical member 23 that is received in the second lens barrel 13 and a prism 24 that is provided at the image-side end portion of the second lens barrel 13, and the image pickup element 7 is held by the second lens barrel 13 through the prism 24. The prism 24 bends incident light at an angle of about 90° and makes the incident light be incident on the image pickup element 7. The prism 24 may be omitted, and the image pickup element 7 may be disposed at the image-side end portion of the second lens barrel 13 so as to be substantially perpendicular to the optical axis A.

A fitting portion 30 is provided at the image-side end portion of the first lens barrel 11, and a fitting portion 31 to which the fitting portion 30 of the first lens barrel 11 is fitted in the direction of the optical axis of the image pickup optical system 6 is provided at the subject-side end portion of the second lens barrel 13. The fitting portion 30 of the first lens barrel 11 and the fitting portion 31 of the second lens barrel 13 are adapted so that the fitting portion 30 of the first lens barrel 11 is fitted inside and the fitting portion 31 of the second lens barrel 13 is fitted outside when the fitting portion 30 of the first lens barrel 11 and the fitting portion 31 of the second lens barrel 13 are fitted to each other.

The second lens barrel 13 includes a contact portion 32 that comes into contact with the optical member 21 provided at the image-side tail of the first optical member group 10 held by the first lens barrel 11. The contact portion 32 is provided around the optical axis A in the fitting portion 31, and comes into contact with the optical member 21 when the fitting portion 30 of the first lens barrel 11 and the fitting portion 31 of the second lens barrel 13 are fitted to each other.

In a state in which the optical member 21 of the first optical member group 10 is in contact with the contact portion 32 of the second lens barrel 13, a space is formed on the image side of the fitting portion 30 of the first lens barrel 11 and the subject side of the fitting portion 31 of the second lens barrel 13 in the direction of the optical axis of the image pickup optical system 6. Accordingly, the first optical member group 10 is positioned with respect to the second optical member group 12, which is held by the second lens barrel 13, in the direction of the optical axis by the contact between the optical member 21 and the contact portion 32.

Accordingly, the first and second lens barrels 11 and 13 are bonded to each other in a state in which the first optical member group 10 is positioned with respect to the second optical member group 12. For example, an adhesive, which can be released by an external stimulus, such as a solvent, heat, or light, is used for the bonding between the first and second lens barrels 11 and 13. Accordingly, the first lens barrel 11 can be separated from the second lens barrel 13.

In terms of easily releasing and removing an adhesive AD that bonds the first lens barrel 11 to the second lens barrel 13, preferably, the subject-side end portion of the fitting portion 31 to be fitted outside and the outer peripheral surface of the first lens barrel 11 are bonded to each other in the shown example in which the fitting portion 30 of the first lens barrel 11 is fitted into the fitting portion 31 of the second lens barrel 13. The adhesive AD, which is applied over the subject-side end portion of the fitting portion 31 to be fitted outside and the outer peripheral surface of the first lens barrel 11, is exposed to the outside even after bonding, and can be easily removed in comparison with, for example, a case in which the adhesive AD is applied between the inner peripheral surface of the fitting portion 31 and the outer peripheral surface of the fitting portion 30.

In addition, in terms of increasing the bonding strength between the first and second lens barrels 11 and 13, it is preferable that a flange portion 33, which faces the subject-side end of the fitting portion 31 of the second lens barrel 13 with an interval therebetween in the direction of the optical axis of the image pickup optical system 6, is provided on the outer peripheral surface of the first lens barrel 11 and the interval between the subject-side end of the fitting portion 31 and the flange portion 33 is filled with the adhesive AD.

In a case in which a stain or a damage occurs on the objective optical member 20, the adhesive AD is released and removed, the first lens barrel 11 is separated from the second lens barrel 13, and the first optical member group 10 and the first lens barrel 11 are replaced all together. An optical member 21 provided at the image-side tail of a first optical member group 10 held by a new first lens barrel 11 comes into contact with the contact portion 32 of the second lens barrel 13, so that the first optical member group 10 is positioned with respect to the second optical member group 12, which is held by the second lens barrel 13, in the direction of the optical axis of the image pickup optical system 6. Then, the first and second lens barrels 11 and 13 are bonded to each other in a state in which the first optical member group 10 is positioned with respect to the second optical member group 12.

The objective optical member 20 and the optical member 21 of the first optical member group 10 are fixed to the first lens barrel 11 in a state in which the objective optical member 20 and the optical member 21 are in contact with each other in the direction of the optical axis of the image pickup optical system 6, and the first optical member group 10 is positioned with respect to the second optical member group 12 in the direction of the optical axis by the contact between the optical member 21 and the contact portion 32 of the second lens barrel 13. For this reason, a positional relationship between the objective optical member 20 and the optical member 21 of the first optical member group 10 and the second optical member group 12 in the direction of the optical axis is reproduced regardless of the tolerance of the first lens barrel 11. Accordingly, the adjustment of a focus, which accompanies the replacement of the first optical member group 10 and the first lens barrel 11, can be made unnecessary.

Since the adjustment of a focus is unnecessary, the objective optical member 20 and the optical member 21 are bonded to each other and the airtightness of the space S between the objective optical member 20 and the optical member 21 can be ensured. Accordingly, even though the first optical member group 10 and the first lens barrel 11 are replaced, the generation of condensation is suppressed and image pickup performance can be maintained.

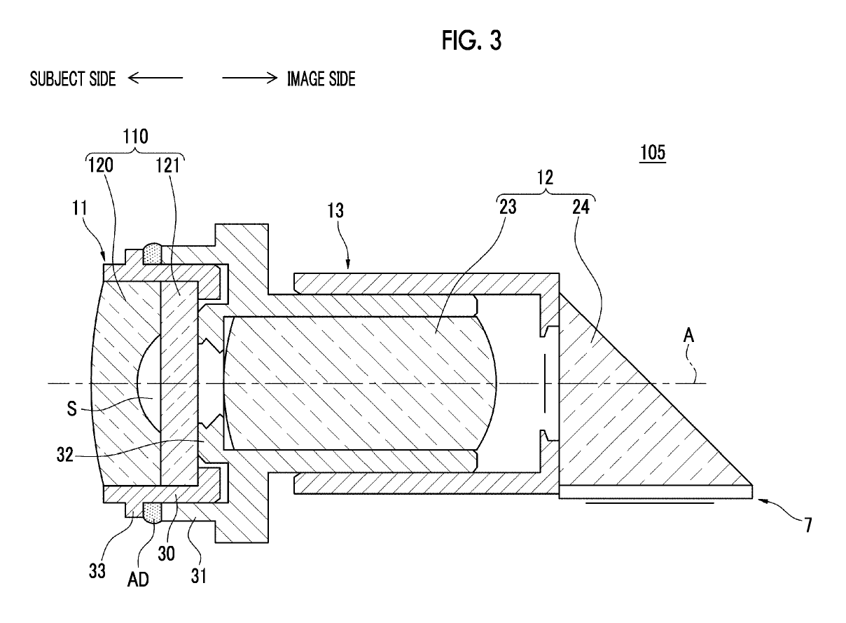

FIG. 3 shows the structure of another example of the image pickup unit of the endoscope that is used to illustrate the embodiment of the invention. Elements, which are common to the above-mentioned image pickup unit 5, will be denoted by common reference numerals and the description thereof will be omitted or simplified.

In an image pickup unit 105 shown in FIG. 3, an objective optical member 120 of a first optical member group 110 and an optical member 121, which is disposed adjacent to the image side of the objective optical member 120, are bonded to each other in a state in which the objective optical member 120 and the optical member 121 are in contact with each other, the outer diameters of the objective optical member 120 and the optical member 121 are integrally machined so that the centers of the objective optical member 120 and the optical member 121 correspond to each other, and all of the objective optical member 120 and the optical member 121 are fitted to the first lens barrel 11 and are held by the first lens barrel 11.

Even in the image pickup unit 105, as in the above-mentioned image pickup unit 5, the objective optical member 120 and the optical member 121 of the first optical member group 110 are fixed to the first lens barrel 11 in a state in which the objective optical member 120 and the optical member 121 are in contact with each other in the direction of the optical axis of the image pickup optical system, and the first optical member group 110 is positioned with respect to the second optical member group 12 in the direction of the optical axis by the contact between the optical member 121 and the contact portion 32 of the second lens barrel 13. Accordingly, the adjustment of a focus, which accompanies the replacement of the first optical member group 110 and the first lens barrel 11, can be made unnecessary.

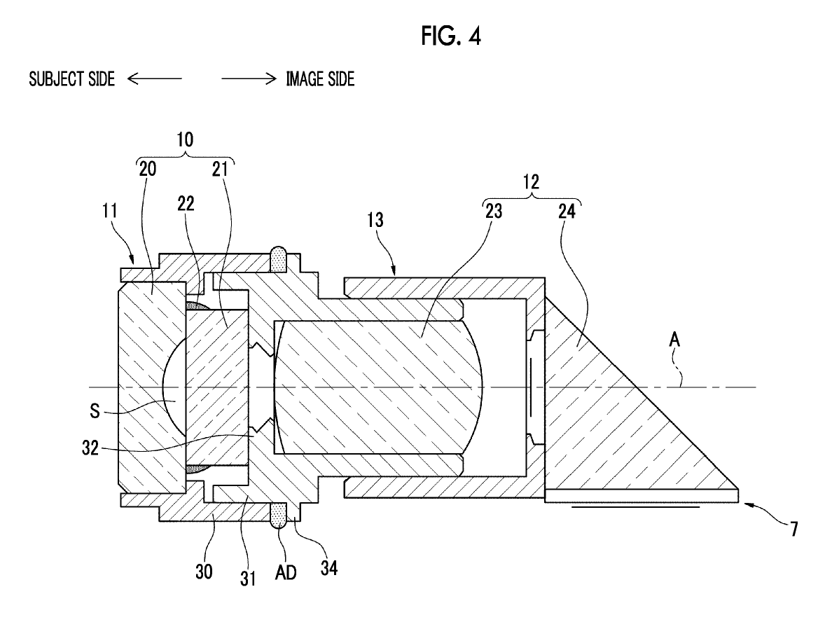

The structure, in which the fitting portion 30 of the first lens barrel 11 and the fitting portion 31 of the second lens barrel 13 are adapted so that the fitting portion 30 of the first lens barrel 11 is fitted inside and the fitting portion 31 of the second lens barrel 13 is fitted outside when the fitting portion 30 of the first lens barrel 11 and the fitting portion 31 of the second lens barrel 13 are fitted to each other, has been described in the above-mentioned image pickup units 5 and 105. However, the fitting portion 30 of the first lens barrel 11 and the fitting portion 31 of the second lens barrel 13 may be adapted so that the fitting portion 30 of the first lens barrel 11 is fitted outside and the fitting portion 31 of the second lens barrel 13 is fitted inside as shown in FIG. 4. In this case, it is preferable that the image-side end portion of the fitting portion 30 to be fitted outside and the outer peripheral surface of the second lens barrel 13 are bonded to each other, a flange portion 34, which faces the image-side end of the fitting portion 30 of the first lens barrel 11 with an interval therebetween in the direction of the optical axis of the image pickup optical system 6, is provided on the outer peripheral surface of the second lens barrel 13, and the interval between the image-side end of the fitting portion 30 and the flange portion 34 is filled with the adhesive AD.

As described above, the image pickup optical system of the endoscope disclosed in this specification includes the first optical member group that includes the plurality of optical members including the objective optical member disposed closest to the subject side, the first lens barrel that holds the first optical member group, the second optical member group that is disposed on the image side of the first optical member group, and the second lens barrel that holds the second optical member group; the plurality of optical members of the first optical member group are in contact with each other in the direction of the optical axis; the first and second lens barrels are fitted to each other in the direction of the optical axis, and the optical member, which is disposed closest to the image side, of the first optical member group comes into contact with the second lens barrel when the first and second lens barrels are fitted to each other; and a space is formed on the image side of the image-side end of the first lens barrel and on the subject side of the subject-side end of the second lens barrel in the direction of the optical axis in a state in which the optical member, which is disposed closest to the image side, of the first optical member group is in contact with the second lens barrel, and the first and second lens barrels are bonded to each other so as to be separable from each other.

Further, in the image pickup optical system of the endoscope disclosed in this specification, the end portion of one lens barrel, which is fitted outside when the first and second lens barrels are fitted to each other, of the image-side end portion of the first lens barrel and the subject-side end portion of the second lens barrel and the outer peripheral surface of the other lens barrel thereof are bonded to each other.

Furthermore, in the image pickup optical system of the endoscope disclosed in this specification, the flange portion, which faces the end portion of one lens barrel with an interval therebetween in the direction of the optical axis, is provided on the outer peripheral surface of the other lens barrel, and the interval between the end portion of one lens barrel and the flange portion is filled with an adhesive.

Moreover, in the image pickup optical system of the endoscope disclosed in this specification, the objective optical member and the optical member, which is disposed adjacent to this optical member, of the first optical member group are bonded to each other so that a space between both the optical members is made airtight.

Further, the image pickup unit of the endoscope disclosed in this specification includes the image pickup optical system of the endoscope and an image pickup element that is provided at the subject-side end portion of the second lens barrel of the image pickup optical system.

Furthermore, the endoscope disclosed in this specification includes the image pickup unit of the endoscope.

According to the invention, there are provided an image pickup optical system and an image pickup unit of an endoscope, and an endoscope of which some optical member groups disposed on the subject side can be replaced and which can reduce time and effort for the adjustment of a focus accompanying the replacement of the optical member groups.

The embodiments of the invention have been descried in detail above. However, the embodiments are merely exemplifications, and the invention may further include various modifications without departing from the scope of the invention. This application is based on JP2014-245927 filed Dec. 4, 2014, the content of which is incorporated herein by reference.

What is claimed is:

1. An image pickup optical system of an endoscope comprising:
a first optical member group that includes an objective optical member disposed closest to a subject side and an image-side optical member that is disposed adjacent to an image side of the objective optical member;
a first lens barrel that holds the first optical member group;
a second optical member group that is disposed on an image side of the first optical member group; and
a second lens barrel that holds the second optical member group,
wherein the objective optical member and the image-side optical member of the first optical member group are in contact with each other in a direction of an optical axis,
the first and second lens barrels are fitted to each other in the direction of the optical axis so that the image-side optical member of the first optical member group is in contact with the second lens barrel, and
a space is provided on an image side of an image-side end of the first lens barrel and on a subject side of a subject-side end of the second lens barrel in the direction of the optical axis in a state in which the image-side optical member of the first optical member group is in contact with the second lens barrel, and the first and second lens barrels are bonded to each other so as to be separable from each other.

2. The image pickup optical system of an endoscope according to claim 1,
wherein an end portion of one lens barrel, which is provided at an outer side when the first and second lens barrels are fitted to each other, of an image-side end portion of the first lens barrel and a subject-side end portion of the second lens barrel and an outer peripheral surface of other lens barrel thereof are bonded to each other.

3. The image pickup optical system of an endoscope according to claim 2,
wherein a flange portion, which faces the end portion of the one lens barrel with an interval therebetween in the direction of the optical axis, is provided on the outer peripheral surface of the other lens barrel, and
the interval between the end portion of the one lens barrel and the flange portion is filled with an adhesive.

4. The image pickup optical system of an endoscope according to claim 1,
wherein the objective optical member and the image-side optical member of the first optical member group are airtightly bonded to each other.

5. The image pickup optical system of an endoscope according to claim 2,
wherein the objective optical member and the image-side optical member of the first optical member group are airtightly bonded to each other.

6. The image pickup optical system of an endoscope according to claim 3,
wherein the objective optical member and the image-side optical member of the first optical member group are airtightly bonded to each other.

7. The image pickup optical system of an endoscope according to claim 1,
wherein a surface, which is in contact with the objective optical member, of the image-side optical member of the first optical member group is a flat surface; and a surface, which is in contact with the second lens barrel, of the image-side optical member of the first optical member group is a flat surface.

8. The image pickup optical system of an endoscope according to claim 2,
wherein a surface, which is in contact with the objective optical member, of the image-side optical member of the first optical member group is a flat surface; and a surface, which is in contact with the second lens barrel, of the image-side optical member of the first optical member group is a flat surface.

9. The image pickup optical system of an endoscope according to claim 3,
wherein a surface, which is in contact with the objective optical member, of the image-side optical member of the first optical member group is a flat surface; and a surface, which is in contact with the second lens barrel, of the image-side optical member of the first optical member group is a flat surface.

10. The image pickup optical system of an endoscope according to claim 4,
wherein a surface, which is in contact with the objective optical member, of the image-side optical member of the first optical member group is a flat surface; and a surface, which is in contact with the second lens barrel, of the image-side optical member of the first optical member group is a flat surface.

11. The image pickup optical system of an endoscope according to claim 5,
wherein a surface, which is in contact with the objective optical member, of the image-side optical member of the first optical member group is a flat surface; and a surface, which is in contact with the second lens ban⁻el, of the image-side optical member of the first optical member group is a flat surface.

12. The image pickup optical system of an endoscope according to claim 6,
wherein a surface, which is in contact with the objective optical member, of the image-side optical member of the first optical member group is a flat surface; and a surface, which is in contact with the second lens barrel, of the image-side optical member of the first optical member group is a flat surface.

13. An image pickup unit of an endoscope comprising:
the image pickup optical system according to claim 1; and
an image pickup element that is provided at an image-side end portion of the second lens barrel of the image pickup optical system.

14. An endoscope comprising:
the image pickup unit according to claim 13.
